(19) United States
(12) Patent Application Publication
(10) Pub. No.: US 2018/0228681 A1
(43) Pub. Date: Aug. 16, 2018

(54) APPARATUS AND METHOD FOR EMITTING LIGHT AND VIBRATION TO A BODY OF A USER

(71) Applicant: JESSICA IVERSON, Rochester, VT (US)

(72) Inventor: JESSICA IVERSON, Rochester, VT (US)

(21) Appl. No.: 15/939,448

(22) Filed: Mar. 29, 2018

Related U.S. Application Data
(63) Continuation-in-part of application No. 15/710,785, filed on Sep. 20, 2017.

(51) Int. Cl. A61H 1/00; A61N 5/06

(52) U.S. Cl. CPC A61H 1/00; A61N 5/0625; A61N 2005/0652; A61N 2005/0645; A61N 2005/0659; A61H 2205/083; A61H 2207/00; A61N 2005/0626; A61N 2005/0663; A61H 2201/165; A61H 2201/1645; A61H 2201/10

(57) ABSTRACT

A light emitting apparatus for use in selected areas of a body of a user, comprising: a flexible wrap; a plurality of lights arranged on the wrap, the wrap is configured to be wrapped around at least one selected area of the body such that the lights face the at least one selected area of the body, wherein each of the plurality of lights are configured to emit a light at a predetermined wavelength to the at least one selected area to perforate one or more fat cells present at the at least one selected area to drain content of the one or more fat cells when a power is supplied to the plurality of lights, the predetermined wavelength is between a range of 630 nm and 635 nm; and a vibration-generating module configured to generate a vibration for stimulating the at least one selected area of the body.

### CROSS REFERENCE WITH RELATED APPLICATION

This is a CIP application of a non-provisional application Ser. No. 15/710,785, filed on 20 Sep. 2017, having title “APPARATUS AND METHOD FOR EMITTING LIGHT TO A BODY OF A USER”.

### FIELD OF THE INVENTION

The present disclosure relates to the field of light emitting devices for use with a body of a user. In particular, the present disclosure is related to a light emitting apparatus for use in selected areas of a body of a user, comprising a flexible wrap comprising lights for emitting light at a predetermined wavelength to areas of the body to increase lipolysis of the fat cells at the areas of the body.

### BACKGROUND

Known light emitting diode (LED) devices may emit infrared and near infrared light of a specified wavelength that can provide therapeutic effects to persons exposed to the light under certain conditions. Other light devices may include the use of low-level laser (LLT) therapy, which also involves exposing body tissues of persons to red and near infrared light. Exposure to the above type of light under certain conditions can alleviate various effects that sun exposure, gravity, pollution, and harsh soaps and chemicals have on the skin. The devices may take the form of a flexible pad having an array of LEDs or other lights on the pad that can emit the infrared light on a user's body. For example, these devices may come in the form of a flexible pad that can be secured to the back, leg, shoulder, elbow or other parts of a user's body to temporarily reduce pain from arthritis, trauma, and strains.

Light devices may benefit from improvements.

### SUMMARY

An embodiment of the present disclosure provides a light emitting apparatus for use in selected areas of a body of a user. The light emitting apparatus comprising a flexible wrap comprising one or more ends configured to detachably connect together. The light emitting apparatus further includes a plurality of lights arranged on an inner surface of the flexible wrap, the flexible wrap is configured to be wrapped around at least one selected area of the body such that the plurality of lights face the at least one selected area, wherein the plurality of lights are configured to emit a light at a predetermined wavelength to the at least one selected area to perforate one or more fat cells present at the at least one selected area and to drain content of the one or more fat cells when a power is supplied to the plurality of lights. The light emitting apparatus further includes a vibration-generating module configured to generate a vibration for stimulating the at least one selected area of the body.

According to an aspect of the present disclosure, the plurality of lights may include a plurality of Light Emitting Diodes (LEDs). Each of the plurality of LEDs is configured to emit an infrared radiation at the predetermined wavelength to the at least one selected area of the body for perforating the one or more fat cells at the at least one selected area for draining the of the content of the one or more fat cells when the power is supplied to the plurality of LEDs.

According to another aspect of the present disclosure, the predetermined wavelength is between a range of 630 nm and 635 nm.

According to another aspect of the present disclosure, the apparatus further includes a switch configured to enable an activation of the plurality of lights to emit a light at the predetermined wavelength to the at least one selected area of the body.

According to another aspect of the present disclosure, the plurality of lights is arranged in an array of lights consisting of 30 columns and 10 rows.

According to another aspect of the present disclosure, the apparatus further includes a timer circuit configured to enable the plurality of lights to emit the light for one second after every four seconds.

According to another aspect of the present disclosure, the vibration-generating module generates the vibration simultaneously with the emission of the light, by the plurality of lights, at the at least one selected area.

Another embodiment of the present disclosure provides a light emitting apparatus for use in selected areas of a body of a user. The light emitting apparatus comprises a flexible wrap comprising one or more ends configured to detachably connect together. The light emitting apparatus further comprises a plurality of lights arranged on an inner surface of the flexible wrap. The flexible wrap is configured to be wrapped around at least one selected area of the body such that the plurality of lights face the at least one selected area. Further, the plurality of lights are configured to emit a light at a predetermined wavelength at the at least one selected area to perforate one or more fat cells present at the at least one selected area and to drain content of the one or more fat cells when a power is supplied to the plurality of lights. The predetermined wavelength is between a range of 630 nm and 635 nm. The light emitting apparatus also includes a vibration-generating module configured to generate a vibration for stimulating the at least one selected area of the body. Further, the vibration-generating module generates the vibration simultaneously with the emission of the light, by the plurality of lights, at the at least one selected area.

Yet another embodiment of the present disclosure provides a method comprising: wrapping a flexible wrap comprising a plurality of lights around at least one selected area of a body of a user such that the plurality of lights face the at least one selected area. The method also includes supplying power to the flexible wrap to cause each of the plurality lights to emit a light at a predetermined wavelength to the at least one selected area to perforate one or more fat cells at the at least one selected area to drain a content of the one or more fat cells, wherein the first predetermined wavelength is generally between 630 nm and 635 nm. The method further includes generating a vibration for stimulation of the at least one selected area of the body.

According to another aspect of the present disclosure, the flexible wrap is wrapped around the at least one selected area by connecting one or more ends of the flexible wrap together.

According to another aspect of the present disclosure, the plurality of lights may include a plurality of Light Emitting Diodes (LEDs), wherein supplying power to the flexible wrap enables the plurality of LEDs to emit infrared radiation at the predetermined wavelength to the at least one selected area of the body to perforate the one or more fat cells to drain the content of the one or more fat cells.

According to another aspect of the present disclosure, the method further includes selecting a switch present on the flexible wrap for activating the plurality of lights to emit the light at the predetermined wavelength to the at least one selected area.

According to another aspect of the present disclosure, the method further includes generating vibration simultaneously with the emission of the light at the at least one selected area.

According to another aspect of the present disclosure, the method further includes setting a timer for setting a duration for at least one of emitting the plurality of lights and generating the vibration for stimulating the at least one selected area of the body.

In another embodiment, an apparatus is provided. The apparatus includes a flexible wrap and a plurality of lights arranged on the wrap. The wrap is configured to wrap around a body of a user such that the lights face selected areas of the body. The apparatus is configured to cause the lights to emit light at a first predetermined wavelength to the selected areas of the body that is sufficient to perforate one or more fat cells at the selected areas of the body to drain the content within the one or more fat cells when power is supplied to the lights. The apparatus is configured to cause the lights to emit light at a second predetermined wavelength to the selected areas of the body at the same time as the lights emit light at the first predetermined wavelength when power is supplied to the lights.

In yet another embodiment, a method is provided. The method includes wrapping a wrap assembly around a body of a user such that a plurality of lights on the wrap face selected areas of the body, and supplying power to the wrap to cause the lights to emit light at a first predetermined wavelength to the selected areas of the body that is sufficient to perforate one or more fat cells at the selected areas of the body to drain the content within the one or more fat cells and simultaneously emit light at a second predetermined wavelength to the selected areas of the body to increase circulation at the selected areas of the body.

In the light emitting apparatus of the present disclosure, vibration may be generated and the light may be emitted simultaneously to a selected area of a body of the user.

In the light emitting apparatus of the present disclosure, vibration may be generated at a different time than the light emitted to a selected area of a body of the user.

Other embodiments of the disclosed apparatus and method emitting light to a body of a user will become apparent from the following detailed description, the accompanying drawings and the appended claims.

### BRIEF DESCRIPTION OF THE DRAWINGS

FIG. 1 is perspective view of the apparatus according to an embodiment of the invention;

FIG. 2 is a perspective view of the apparatus of FIG. 1 showing the wrap wrapped around the body of a user;

FIG. 3 is a front view of the wrap of the apparatus of FIG. 1 with the sheet removed for illustrative purposes;

FIG. 4 is a block diagram of the components of the apparatus of FIG. 1;

FIG. 5 is a block diagram of the first and second integrated circuits connected to the LEDs of the apparatus of FIG. 1;

FIG. 6 is a top view of chips according another embodiment of the apparatus of FIG. 1;

FIG. 7 is a top perspective view of a chip of FIG. 6;

FIG. 8 is a flow diagram of a method for emitting light to a body of a user using the apparatus of FIG. 1;

FIG. 9 is a perspective view of a light emitting apparatus showing a flexible wrap wrapped around a body of a user;

FIG. 10 is a block diagram of the components of the light emitting apparatus of FIG. 9; and

FIG. 11 is a flow diagram of a method for emitting light to a body of a user using the light emitting apparatus of FIG. 9.

### DETAILED DESCRIPTION

It will be readily understood that the components of the embodiments as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

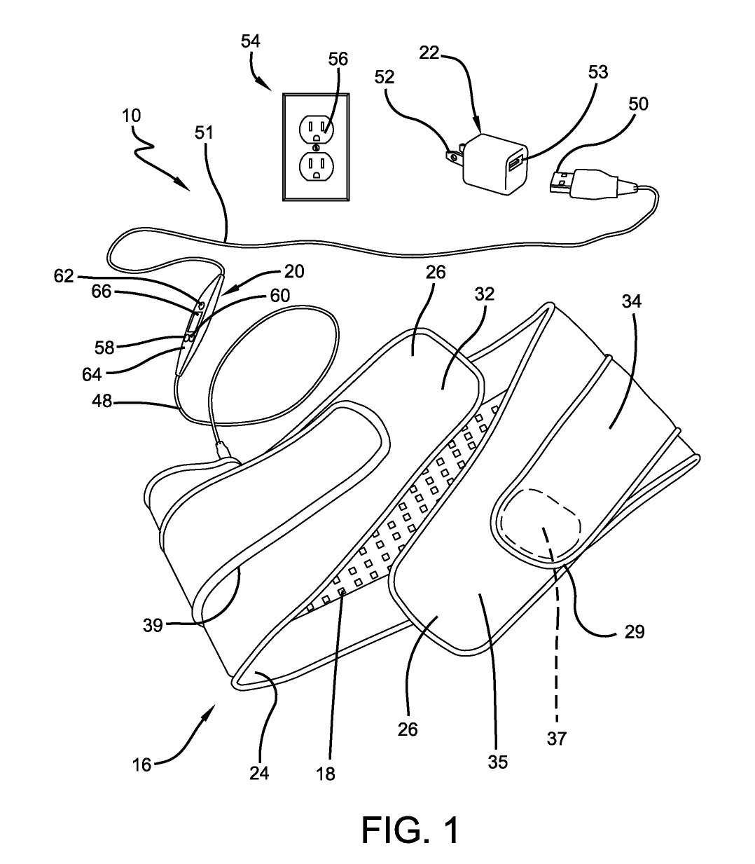

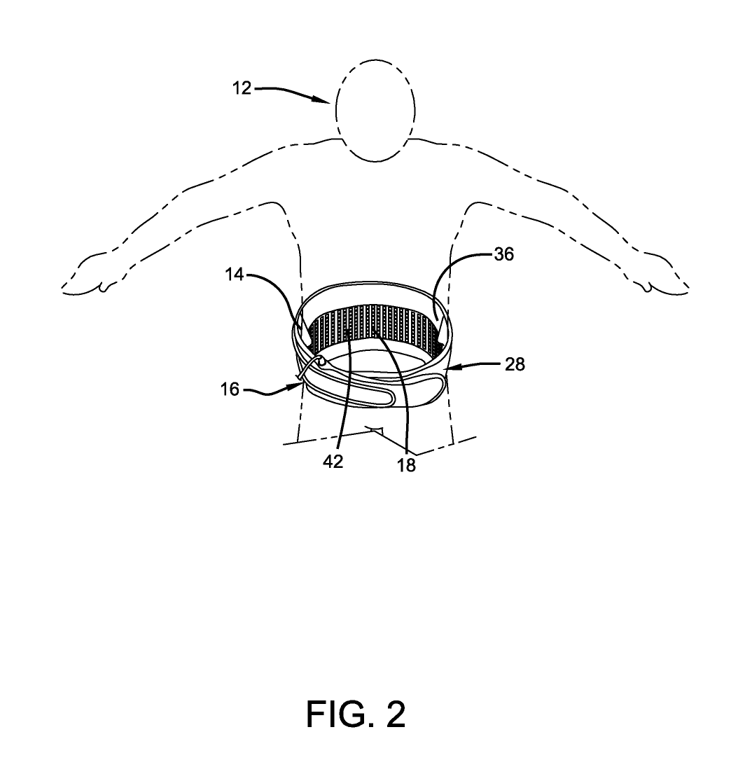

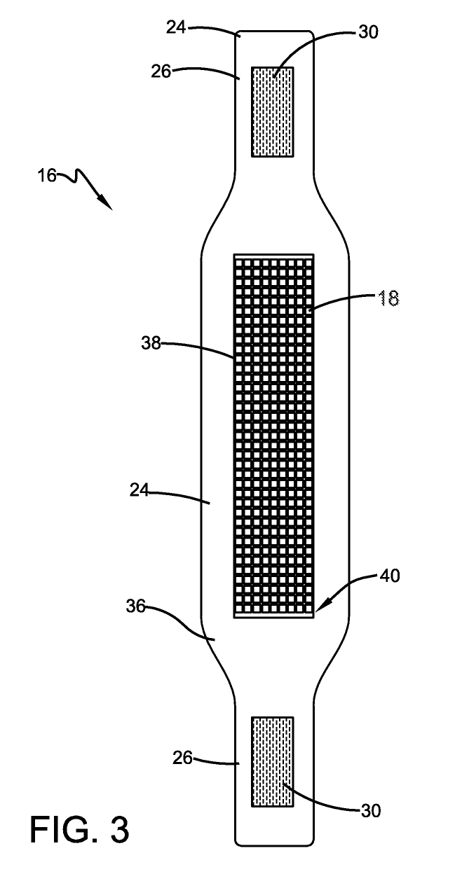

FIGS. 1-6 illustrate an embodiment of an apparatus 10 and method for emitting light to a body 14 of a user 12. Referring to FIG. 1, the apparatus 10 comprises a flexible elongated wrap 16, a number of light emitting diode (LED) lights 18, a switch controller 20, and a power adapter 22. The wrap 16 may be a silica fiber cloth or other suitable main body belt. The wrap 16 is elongated and may be 116 cm in length. The wrap 16 includes a central body portion 24 and opposite arms 26 that extend axially from the body portion 24. The wrap 16 includes a hook and pile fastener 28 (FIGS. 2 and 3) such as Velcro® thereon to detachably attach the arms 26 or other parts of the wrap 16 together when the wrap is wrap around an object. In particular, a first lineal fabric strip 30 comprising small hooks is sewn or otherwise adhered to one or each arm 26 at the front side 36 of the wrap 16 and a second lineal fabric strip 32 (FIG. 1) comprising smaller loops is sewn or otherwise adhered to the rear side 35 of the wrap 16. When the strips 30, 32 are pressed together, the hooks catch in the loops and the two arms 26 fasten or bind temporarily together. A looped fabric strip defining a handle 34 (FIG. 1) is attached to the rear side 35 of the wrap 16 and extends from one arm 26 along the body portion 24 of wrap 16 to the other arm 26. The looped fabric strip 34 may be detachably attached to the arms 26 at the rear side 35 of the wrap 16 by a hook and pile fastener 29 (FIGS. 1 and 2) such as Velcro®. In particular, referring to FIG. 1, a third lineal fabric strip 37 comprising small hooks is sewn or otherwise adhered to the front side 39 of the handle 34 at one or each axial end of the handle 34. When the third and second strips 37, 32 are pressed together, the hooks catch in the loops and the handle 34 and wrap 16 are fasten or bind temporarily together. Alternatively, the looped fabric strip 34 may be sewn or otherwise secured at its axial ends to their respective arms 26.

As illustrated in FIG. 3, the body portion 24 has a rectangular recess 38 at the front side 36 of the wrap 16. The surface 40 of the recess 38 may comprise a uniform color such as black. The surface may alternatively comprise a reflective color surface such as a mirrored surface. Other alternative embodiments may have the surface 40 be comprises of a non-mirrored uniform color or non-uniformly colored. The apparatus 10 includes an array of the lights 18 mounted on the surface 40 of the recess 38 or otherwise secured to the wrap 16 at the location of the recess 38. The lights 18 may be covered by a flexible sheet 42 (FIG. 2) made of plastic or other suitable material. The sheet 42 may be transparent and extend over the recess 38 to allow the light emitted by the lights 18 to shine through. The lights 18 may comprise infrared LEDs. The apparatus 10 may comprise 300 LEDs arranged as 30 columns of LEDs with each column including 10 rows of LEDs. Alternative embodiments may include additional rows and/or columns of LEDs. Alternative embodiments may also include less LEDs. Additional embodiments may employ near infrared LEDs in addition to, or in place of, any of the infrared LEDs.

One embodiment uses the specific configuration and number of LEDs in order to provide the level of infrared light necessary to perforate one or more fat cells at the selected areas of the body to drain the content within the one or more fat cells to enhance fat loss and detoxification of the user's body. This level of infrared light also increases the lipolysis of the fat cells at the areas of a user's body 14 facing the LEDs when the wrap 16 is wrapped around the body 14. The current embodiment also uses the specific configuration and number of LEDs in order to provide the level of infrared light necessary to reduce pain and inflammation at the areas of the body. This level of infrared light also boosts circulation of the body and penetrates deeply into the body. This level of infrared light also stimulates production of nitric oxide in the blood, which opens up the capillaries of the user's body, thus increasing the blood flow. This level of infrared light also creates a metabolic effect to burn calories. Alternative embodiments may include non-infrared LEDs. The power for the apparatus is between 5 watts and 20 watts during operation of the apparatus 10. In one example, power for the apparatus is 20 watts during operation of the apparatus. The heat generated by the LEDs may cause the temperature of the space outwardly adjacent the LEDs to be 104 degrees. The recess 38 defines the treatment area of the body of the user. In one example, the treatment area is 18 inches by 5 inches. In another example, the treatment area may be 19.7 inches by 4.3 inches (11 cm by 50 cm).

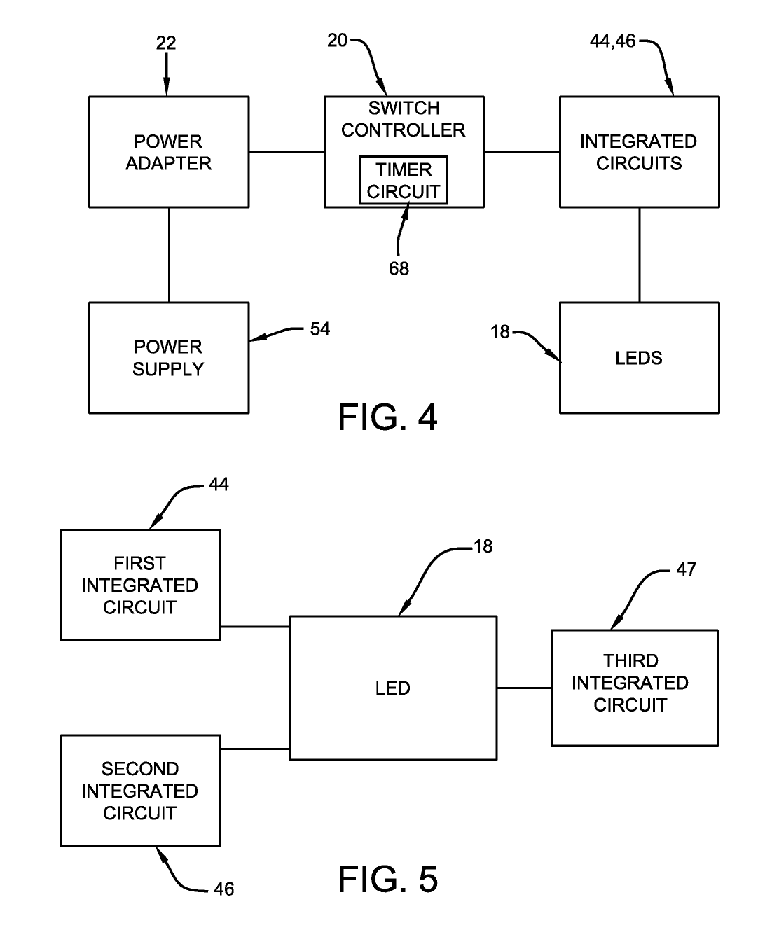

As illustrated in FIGS. 4 and 5, each of the LEDs may be electrically coupled to first and second circuits 44, 46. The first and second circuits may be first and second integrated circuits or chips 44, 46. The first and second integrated circuits 44, 46 may be mounted on a printed circuit board or other suitable platform. The printed circuit board may be flexible. The output of the switch controller 20 is electrically coupled to the first and second integrated circuits 44, 46 via an electric cord 48 (FIG. 1). The input of the switch controller 20 is electrically coupled to the power adaptor 22 via an electric cord 51. In particular, the free end of the electric cord comprises a USB connector 50 that may be plug into a USB port 53 of the power adapter 22 as illustrated in FIG. 1. Alternatively, the power adapter 22 may not have a USB port and the end of the electric cord may not have the USB connector and instead may be electrically coupled to the power adapter 22. The power adapter 22 includes plugs 52 that may be plugged into slots 56 of an electrical outlet 54 or other power source that supplies between 100-240 VAC at 50/60 hertz. For example, the power adapter 22 may be plugged into an electrical outlet that supplies 120 VAC at 60 hertz. The power ranking of the apparatus 10 may be 1, 2, 3, 4, or 5. Alternatively, instead of the electrical outlet 54 and power adapter 22, the power source 54 may be a battery that supplies a 12-volt DC.

The first integrated circuit 44 is configured to cause the LEDs 18 to emit infrared radiation at a first predetermined wavelength to the selected areas of the body that is sufficient to perforate one or more fat cells at the selected areas of the body to drain the content within the one or more fat cells. This emitted infrared radiation at the first predetermined wavelength also increases the lipolysis of the fat cells at the areas of a user's body 14 facing the LEDs when the wrap 16 is wrapped around the body 14. The first predetermined wavelength may be between 630 nm and 635 nm. In one exemplary embodiment, the wavelength may be 635 nm.

The second integrated circuit 46 is configured to cause the LEDs 18 to emit infrared radiation at a second predetermined wavelength that reduces pain and inflammation at the areas of the body facing the LEDs when the wrap 16 is wrapped around the body 14. This emitted infrared radiation at the second predetermined wavelength boosts circulation of the body and penetrates deeply into the body. This emitted infrared radiation at the second predetermined wavelength stimulates production of nitric oxide in the blood, which opens up the capillaries of the user's body, thus increasing the blood flow. This emitted infrared radiation at the second predetermined wavelength also creates a metabolic effect to burn calories. The second predetermined wavelength may be between 830 nm and 880 nm. In one exemplary embodiment, the second predetermined wavelength may be 850 nm. The emitted radiation may be FAR-Infrared in 850 nm wavelength.

The apparatus may optionally include a third circuit 47. The third circuit may be a third integrated circuit 47 as seen in FIG. 5. Each of the LEDs may be electrically coupled to third integrated circuit 47. The third integrated circuit 47 may be mounted on a printed circuit board or other suitable platform. The printed circuit board may be flexible. The output of the switch controller 20 is electrically coupled to the third integrated circuit 476 via the electric cord 48. The third integrated circuit 47 is configured to cause the LEDs 18 to emit infrared radiation at a third predetermined wavelength that increase collagen formation in the selected areas of the body at the areas of the body facing the LEDs when the wrap 16 is wrapped around the body 14, which helps speed up the healing of injuries such as cuts. This emitted infrared radiation at the third predetermined wavelength also boosts cellular energy by stimulating Adenosine triphosphate (ATP). This emitted infrared radiation at the third predetermined wavelength also penetrates up to ¾ of an inch into the skin and helps to flatten the skin and smooth skin wrinkles. In one exemplary embodiment, the second predetermined wavelength may be 660 nm. The emitted radiation may be near infrared in the 660 nm wavelength. Infrared radiation at this wavelength may also be visible to the user. First, second, and third integrated circuits may be separate integrated circuits or chips, or circuits that are provided on a single chip.

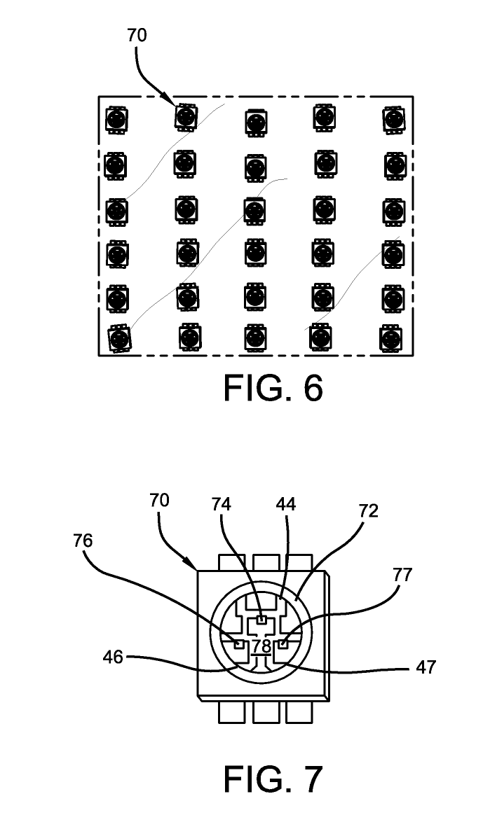

FIGS. 6 and 7 show exemplary chips 70 for the LEDs 18. As illustrated in FIG. 7, each chip 70 comprises the first, second, and third circuits 44, 46, and 47. A mounting recess 72 is formed in the top of the chip and securely receives an LED 18. First, second, and third output ports 74, 76, 77 are provided on the bottom side 78 of the recess 72. The output of the switch controller 20 is electrically coupled to the chips 70 via the electric cord 48. The output signals of the first, second, and third circuits 44, 46, 47 are outputted through their corresponding first, second, and third output ports 74, 76, 77. The output signals cause the LED 18 to emit radiation at the area of the LED 18 adjacent or otherwise corresponding to respective out ports. This enables the LED 18 to emit radiation in three wavelengths (e.g. 635 nm, 660 nm, and 850 nm) at the same time.

Alternatively or in addition, the first and/or second and/or third and/or additional integrated circuits or circuits may be configured to cause the LEDs to emit light at wavelengths between 307 nm and 347 nm (purple light spectrum), 620 nm and 638 nm (red light spectrum), 415 nm and 475 nm (blue light spectrum), and 575 nm and 620 nm (yellow light spectrum).

The switch controller 20 may have three settings or modes that enable a user to select which function or treatment that the wrap 16 performs. The switch controller 20 may be a variety of types. For example, as illustrated in FIG. 1, the switch controller 20 may be membrane touch switch that has first, second, and third push buttons 58, 60, 62 that are each pressed by a finger of the user 12 to select the desired mode of operation of the apparatus 16. The switch controller 20 may include a plastic housing 64 that houses the electronic components of the switch controller 20. The switch controller 20 may include a display 66 to indicate the mode of operation and other information such as the time. Alternatively, the switch controller 20 may include a touch screen for the user to select the desired mode of operation. In another example, the switch controller may be configured to enable a user to select the desired mode of operation by a cell phone or a remote controlled device. The first push button 58 may be pressed or otherwise activated to place the apparatus 16 in the weight loss mode in which the LEDs 18 first predetermined wavelength to the selected areas of the body that is sufficient to perforate one or more fat cells at the selected areas of the body to drain the content within the one or more fat cells and also increases the lipolysis of the fat cells at the areas of a user's body 14 facing the LEDs when the wrap 16 is wrapped around the body 14.

The second push button 60 may be pressed or otherwise activated to place the apparatus in a pain therapy mode in which the LEDs 18 emit infrared radiation at a wavelength that reduces pain and inflammation at the areas of the body facing the LEDs when the wrap 16 is wrapped around the body 14. The third button 62 may be pressed or otherwise activated to place the apparatus in the visible light mode. In the visible light mode, the LEDs 18 emit infrared radiation at a third predetermined wavelength that increase collagen formation in the selected areas of the body at the areas of the body facing the LEDs when the wrap 16 is wrapped around the body 14. Also, in the visible mode, non-infrared LEDs emit light at a wavelength of approximately 660 nm. Non-infrared light of this wavelength is useful because (i) light of this wavelength is within the visible spectrum, so the user knows the device is powered on and working; and (ii) the wavelength of light may possess therapeutic qualities for skin. It should be noted that any non-infrared LEDs that produce visible light may be utilized in place of the 660 nm LEDs for informing the user that the unit is currently active and operational. A fourth button may be added to the switch controller 20 or another suitable way may be used to activate a timer that allows operation of the wrap 16 for a predetermined time. The time may be 10, 20, 30, 40, 50 or 60 minutes. The switch controller may have another push button or other suitable way to place the switch controller in a more aggressive pain therapy mode. Alternatively, the fourth push button may be used for a different setting or be eliminated from the switch controller. It is further contemplated that in other variations LEDs may be used that produce infrared light in any part of the infrared spectrum.

Alternatively or in addition, the switch controller 20 may be configured to have one push button that will cycle through the three modes and set the time of operation. For example, the user may press the first push button 58 of the switch controller 20 once to place the switch controller 20 in the weight loss mode. The user then may then press the first push button 58 a second time to place the switch controller 20 in the pain therapy mode. A user may then press the first push button 58 a third time to place the switch controller in the visible light mode. A user may then press the first push button 58 a fourth time and hold it down for a number of seconds to set the wrap 16 to operate for a first predetermine time. If the user does not want the wrap 16 to operate at the first predetermined time, a user may then press the first push button 58 a fifth time and hold it down for a number of seconds to set the wrap 16 to operate for a second predetermine time. Further times of pressing of the first push button 58 and holding it down for a number of seconds may set the wrap 16 to operate for other selected predetermine times. When at the portion of the cycle that sets the predetermine time of operation of the wrap 16, pressing the first push button 58 and not holding it down for a number of seconds will not select the corresponding predetermine time of operation of the wrap, but enable the user to select the next predetermined time. After the user presses the push button 58 the last time to select a predetermine time of operation of the wrap 16, or cycle past the last predetermine time of operation, the cycle begins again to enable the user to select the three modes of operation beginning with the weight loss mode.

The switch controller may also be configured in combination with the chips of FIGS. 6 and 7 to enable operation of the weight loss, pain therapy, and visible light modes at the same time such that one or more or all of the LEDs emit radiation of the three different wavelengths at the same time. Alternatively or in addition, the switch controller or may also be configured in combination with the chips 70 of FIGS. 6 and 7 to enable operation of a combination of two of the modes of operation at the same time such that one or more or all of the LEDs emit radiation of the selected two different wavelengths at the same time. For example, the first push button may be pressed a fourth time to have all three modes operate at the same time, a fifth time to have just the weight loss and pain therapy mode operate at the same time, a sixth time to have just the pain therapy mode and visible light mode operate at the same time, and a seventh mode to have just the weight loss and visible light mode operate at the same time. A user may press the first push button an eighth time to start the cycle portion to select the predetermined time of operation of the wrap 16.

The switch controller 20 may be configured to enable the user 12 to select via a push button or other suitable selector a first group of LEDs 18 to emit light at the first predetermined wavelength (e.g. 635 nm) to the selected areas of the body facing the first group of LEDs that is sufficient to perforate one or more fat cells at the selected areas of the body to drain the content within the one or more fat cells when the wrap is placed at that position by a user 12. The switch controller 20 may be configured to enable the user 12 to select via a push button or other suitable selector a second group of LEDs 18 to emit light at the second predetermined wavelength (e.g. 850 nm) that reduces pain and inflammation at the selected areas of the body 14 facing the second group of LEDs when the wrap 16 is placed at that position by a user 12. The switch controller 20 may be configured to enable the user 12 to select via a push button or other suitable selector a third group of LEDs 18 to emit light at the third predetermined wavelength (e.g. 660 nm). In one exemplary embodiment, the user may select 150 LEDs as the first group and 150 LEDs as the second group. The first, second, and third groups of LEDs may all operate to emit infrared radiation at the same time or just one or combination of two of the first, second, and third groups may operate to emit infrared radiation.

Alternatively or additionally, the apparatus may be configured to enable the wrap 16 to operate the first group of LEDs or a predetermined number of LEDs in the first group at one or more of the modes of operation for a predetermined time and/or enable the wrap 16 to operate the second group of LEDs or a predetermined number of LEDs in the second group at one or more of the modes of operation for a predetermined time and/or to enable the wrap 16 to operate the third group of LEDs or a predetermined number of LEDs in the third group at one or more of the modes of operation for a predetermined time. Alternatively or additionally, the apparatus may be configured to enable the wrap 16 to operate at one or more modes of operation for a predetermined time and then operate at a different mode or combination of modes for a predetermined time without the user having to select the different mode or combination of modes of operation.

The switch controller 20 may include a timer circuit 68 (FIG. 4) that modulates the infrared light emitted by the LEDs 18 such that LEDs 18 turn off the light for one second for every four seconds. This increases the user's body cells ability to respond to the infrared radiation. Alternatively, the timer circuit 68 may be a separate element located outside of the switch controller 20.

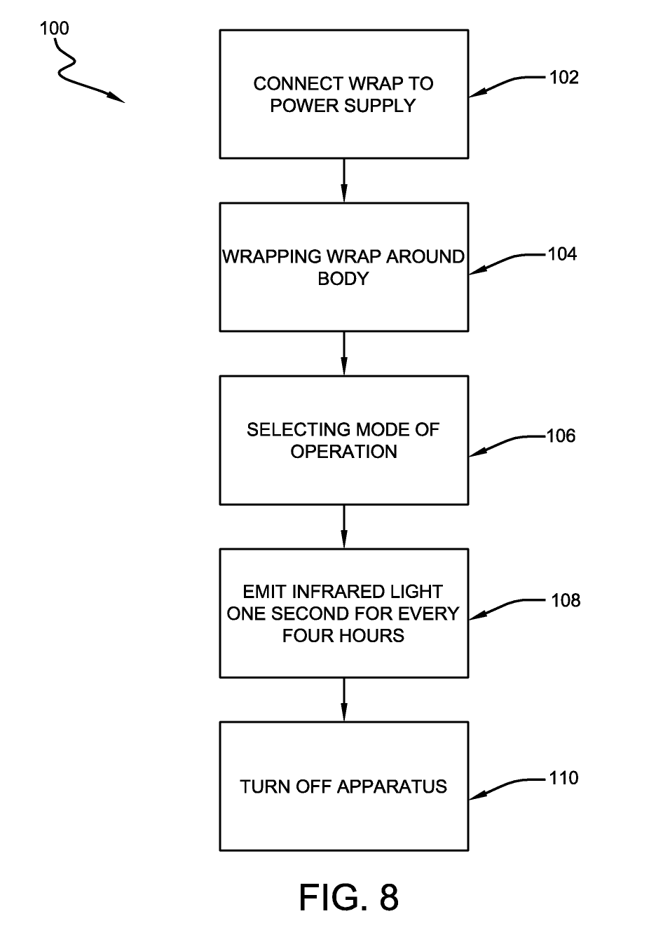

FIG. 8 illustrates an exemplary method 100 for using and operating the apparatus. In step, 102, the power adapter 22 is plugged into the electrical outlet 54 and the USB connector 50 is plugged into the USB port 53 of the power adapter 22. In step 104, the user 12 grasps the handle 34 and wraps the wrap 16 around the body 14 of the user 12 such that front side 36 of the wrap 16 faces the body of the user as shown in FIG. 2. This step includes pressing the lineal fabric strips 30, 32 together to detachably attach the arms 26 of the wrap together. In step 106, the user selects the mode of operation. For example, the user 12 may press the first push button 58 of the switch controller 20 to place and operate the apparatus 16 in the weight loss mode. The user 12 may also select the time that the LEDs 18 operate to emit the infrared radiation. When the apparatus 16 is operated in the weight loss mode, power is supplied from the electrical outlet 54 into the power adaptor 22, where it is converted into a 12 volt DC. The 12-volt DC is inputted into the input of the switch controller 20. In the weight loss mode, the switch controller 20 directs the 12-volt DC through each first integrated circuit or circuit 44. Each first integrate circuit 44 or circuit converts 12 volt DC to a first output signal that is inputted to the corresponding LED 18 to cause the LED 18 to emit infrared radiation at a wave length of 635 nm that is sufficient to perforate one or more fat cells at the selected areas of the body 14 to drain the content within the one or more fat cells.

Alternatively, the user 12 may instead press the second push button 60 (or first button 58 a second time for the corresponding embodiment mentioned above) to place the switch controller 20 in the pain therapy mode. In the pain therapy mode, the switch controller 20 directs the 12-volt DC through each second integrated circuit or circuit 46. Each second integrate circuit or circuit 46 converts the 12 volt DC to a second output signal that is inputted to the corresponding LED 18 to cause the LED 18 to emit infrared radiation at a wave length of 850 nm to reduce pain and inflammation at the selected area of the body 14. Alternatively or additionally, the third push button 62 may be pushed by the user to place the switch in the visible light mode. In the visible light mode, the switch controller 20 directs the 12-volt DC through each third integrated circuit or circuit 47. Each third integrate circuit or circuit 47 converts the 12 volt DC to a third output signal that is inputted to the corresponding LED 18 to cause the LED 18 to emit infrared radiation at a wave length of 660 nm. Alternatively, the user 12 may operate the switch controller 20 to select the first, second, and/or third group of LEDs 18, which operate to emit radiation in a manner as previously mentioned. Alternatively, or in addition, the user may operate the switch controller 20 to enable operation of a combination of one two or three of the modes of operation at the same time or other time as previously mentioned. In step 108, the timer circuit 68 is activated to modulate the infrared light emitted by the LEDs 18 such that LEDs 18 turn off their light for one second for very four seconds. In step 110, the apparatus is then turn off to stop the supply of power to the apparatus 16.

Alternatively or in addition, the lights may be laser lights that are configured to emit light of the previously mentioned wavelengths that is sufficient to perforate one or more fat cells at the selected areas of the body to drain the content within the one or more fat cells, or to reduce pain and inflammation at the areas of the body as previously mentioned, or to provide visibility. In all other aspects, the apparatus that uses laser lights would be structured and function as that for the LEDs with the light emitting red and near infrared (NI) light to deliver the same reaction to the tissues of the user's body.

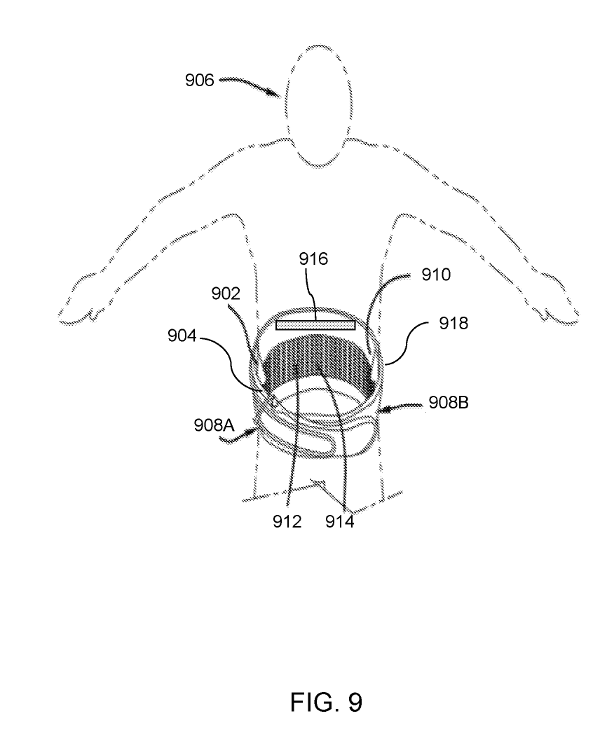

FIG. 9 is a perspective view of a light emitting apparatus 902 showing a flexible wrap 904 wrapped around a body of a user 906. The light emitting apparatus 902 may include a flexible wrap 904 comprising one or more ends 908A-908B and a plurality of lights 912 present on an inner surface 910 of the wrap 904. The flexible wrap 904 may be a silica fiber cloth or other suitable main body belt. The flexible wrap 904 may be an elongated wrap. In some embodiments, a length of the flexible wrap may be 116 cm. The wrap 904 includes a central body portion and opposite arms or the one or more ends 908A-908B that extends axially from the body portion of the flexible wrap. In some embodiments, the flexible wrap 904 may include a hook and pile fastener such as Velcro® thereon to detachably attach the one or more ends 908A-908B or other parts of the flexible wrap 904 together when the flexible wrap is wrapped around an object such as the user 906. In particular, a first lineal fabric strip comprising small hooks is sewn or otherwise adhered to one or more ends 908A-908B at the inner surface 910 of the wrap 904 and a second lineal fabric strip comprising smaller loops may be sewn or otherwise adhered to a rear side of the flexible wrap 904. When the first and second lineal fabric strips are pressed together, the hooks catch in the loops and the one or more ends 908A-908B fasten or bind temporarily together. Further, a looped fabric strip defining a handle may be attached to the rear side of the flexible wrap 904 and may extend from one end along the body portion of the flexible wrap 904 to the other end. The looped fabric strip may be detachably attached to the ends 908A-908B at the rear side of the flexible wrap 904 by a hook and pile fastener such as Velcro®.

Further, the flexible wrap 904 may include a flexible sheet 914 over the plurality of lights 912. The flexible sheet 914 may be of suitable material such as but not limited to fabric, silicon, plastic, and so forth. The flexible sheet 914 may be transparent and extend over the recess to allow the light emitted by the lights 912 to shine through. The lights 912 may comprise infrared LEDs. In some embodiments, the apparatus 902 may comprise 300 LEDs arranged as 30 columns of LEDs with each column including 10 rows of LEDs. Alternative embodiments may include additional rows and/or columns of LEDs. Alternative embodiments may also include less LEDs. Additional embodiments may employ near infrared LEDs in addition to, or in place of, any of the infrared LEDs.

In some embodiments, a specific configuration and number of LEDs or the lights 912 may be used in order to provide the level of infrared light necessary to perforate one or more fat cells at the selected areas of the body of the user 906 to drain the content within the one or more fat cells to enhance fat loss and detoxification of the body of the user 906. This level of infrared light also increases the lipolysis of the fat cells at the areas of a user's body 14 facing the LEDs or the lights 912 when the flexible wrap 904 is wrapped around a selected area 918 on body of the user 906. The current embodiment also uses the specific configuration and number of LEDs in order to provide the level of infrared light necessary to reduce pain and inflammation at the areas 918 of the body. This level of infrared light also boosts circulation of the body and penetrates deeply into the body. This level of infrared light also stimulates production of nitric oxide in the blood, which opens up the capillaries of the body of the user 906, thus increasing the blood flow. This level of infrared light also creates a metabolic effect to burn calories. Alternative embodiments may include non-infrared LEDs. The power for the light emitting apparatus 902 may be between 5 watts and 20 watts during operation of the apparatus 902. In one example, power for the apparatus is 20 watts during operation of the apparatus. The heat generated by the LEDs may cause the temperature of the space outwardly adjacent the LEDs to be 104 degrees. The recess may define the treatment area of the body of the user 906. In one example, the treatment area is 18 inches by 5 inches. In another example, the treatment area may be 19.7 inches by 4.3 inches (11 cm by 50 cm).

Further, the apparatus 902 may include a vibration-generating module 916 configured to generate one or more vibrations for stimulating the selected area 918 of the body of the user 906. The user 906 may control the vibration-generating module 916 by a switch. Further, the user 906 may select a mode on the switch. Examples of the mode may include a light mode, a vibration mode, and a light and vibration mode. In some embodiments, the vibration is generated simultaneously with the emission of the light at the at least one selected area. In alternate embodiments, the vibration is generated at a different time than the light emission by the plurality of lights 912.

Further, the light emitting apparatus 902 may be used to boost system activity to benefit weight loss. The unique wavelength of light emitted from the lights may reach the fat layer and may cause the membranes of the fat cells to alter the permeability of the cell wall. The fat cells walls may open up and dispel wastes from within the cell out into the interstitial layer. The fat is then may be broken down into free fatty acids and glycerol, and enters the blood stream where the body can use it as a fuel or energy or may naturally eliminate in stool and urine via the lymphatic system and liver.

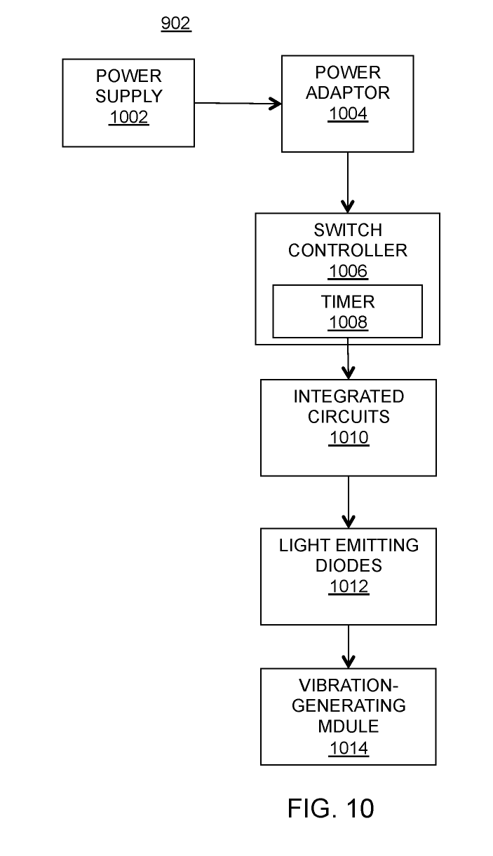

FIG. 10 is a block diagram of the components of the light emitting apparatus 902 of FIG. 9. As shown, the light emitting apparatus 902 includes a power supply 1002, a power adapter 1004, a switch controller 1006 comprising a timer 1008, an integrated circuit 1010, a plurality of light emitting diodes (LEDs) 1012 and a vibration-generating module 1014.

The integrated circuit 1010 may be mounted on a printed circuit board or other suitable platform. The printed circuit board may be flexible. The output of the switch controller 1006 may be electrically coupled to the integrated circuit 1010 via an electric cord. The input of the switch controller 1006 may be electrically coupled to the power adaptor 1004 via an electric cord. In particular, the free end of the electric cord may comprise a USB connector that may be plug into a USB port of the power adapter 1004. Alternatively, the power adapter 1004 may not have a USB port and the end of the electric cord may not have the USB connector and instead may be electrically coupled to the power adapter 1004. The power adapter 1004 may include plugs that may be plugged into slots of an electrical outlet or other power source that supplies between 100-240 VAC at 50/60 hertz. For example, the power adapter 1004 may be plugged into an electrical outlet that supplies 120 VAC at 60 hertz. The power ranking of the apparatus 902 may be 1, 2, 3, 4, or 5. In alternative embodiments, instead of the electrical outlet and power adapter 1004, the power source 1002 may be a battery that supplies a 12-volt DC.

The integrated circuit 1010 is configured to cause the LEDs 1012 to emit infrared radiation at a predetermined wavelength to the selected areas of the selected areas 918 that is sufficient to perforate one or more fat cells at the selected areas 918 of the body to drain the content within the one or more fat cells. This emitted infrared radiation at the predetermined wavelength also increases the lipolysis of the fat cells at the areas of the body of the user 906 facing the LEDs when the flexible wrap 904 is wrapped around the body. The predetermined wavelength may be between 630 nm and 635 nm. In some embodiments, the wavelength may be 635 nm.

The switch controller 1006 may be activated to set the timer 1008 or to control the vibration-generating module 1014 and the LEDs 1012. The user 906 can select a mode by using the switch controller 1006. The mode may be a light mode, a vibration mode, and a light and vibration mode.

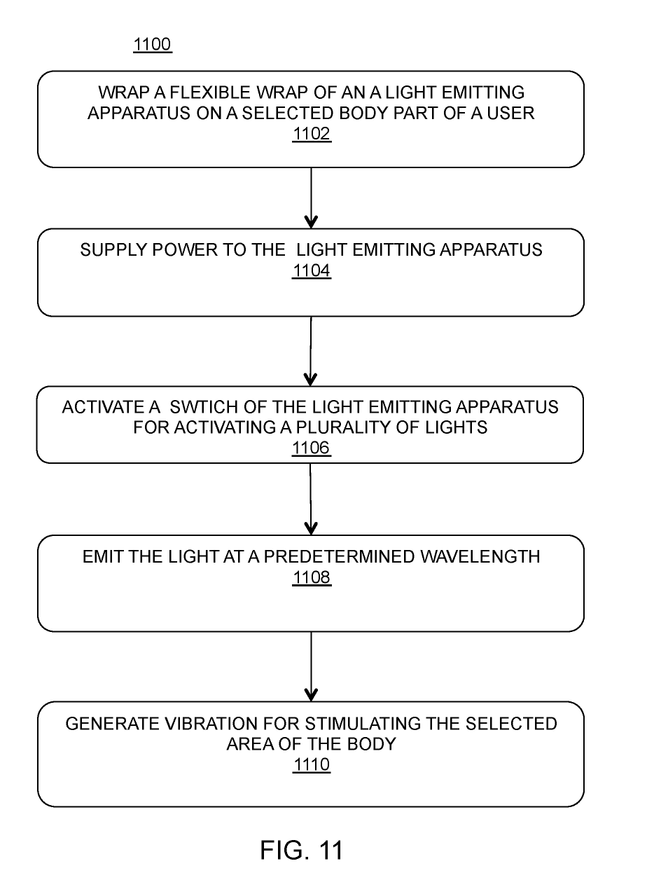

FIG. 11 is a flow diagram of a method 1100 for emitting light to a body of a user using the light emitting apparatus 902 of FIG. 9. As shown, the method 1100 includes wrapping a flexible wrap of the light emitting apparatus 902 on a selected body part of a body of the user 906 at step 1102. At step 1104, power is supplied to the light emitting apparatus. Then at step 1106, a switch of the light emitting apparatus 902 is activated for activating or initiating the plurality of lights. Then at step 1108, the plurality of lights emits the light at a predetermined wavelength. In some embodiments, the predetermined wavelength is between 630 nm and 635 nm. Thereafter, at step 1110, a vibration is generated by a vibration-generating module of the light emitting apparatus 902 for stimulating the selected area 918 of the body of the user 906. In some embodiments, the light and the vibration is generated simultaneously to the selected area 918 of the body of the user 906.

The light emitting apparatus of the present disclosure may be used for reducing the appearance of the cellulite in the user's body.

In the light emitting apparatus of the present disclosure, vibration may be generated and the light may be emitted simultaneously to a selected area of a body of the user.

In the light emitting apparatus of the present disclosure, vibration may be generated at a different time than the light emitted to a selected area of a body of the user.

The light emitting apparatus of the present disclosure may be used for fast and effective lipolysis of the fat cells for fat loss and detoxification.

The light emitting apparatus of the present disclosure may be used for increased collagen formation of skin rejuvenation.

The light emitting apparatus of the present disclosure may be used for increasing skin radiance and skin elasticity.

The light emitting apparatus of the present disclosure may be used to boost system activity to benefit weight loss. The unique wavelength of light emitted from the lights may reach the fat layer and may cause the membranes of the fat cells to alter the permeability of the cell wall. The fat cells walls may open up and dispel wastes from within the cell out into the interstitial layer. The fat is then may be broken down into free fatty acids and glycerol, and enters the blood stream where the body can use it as a fuel or energy or may naturally eliminate in stool and urine via the lymphatic system and liver.

The light emitting apparatus of the present disclosure may be used for pain and inflammation reduction.

The light emitting apparatus of the present disclosure may be used for body contouring.

The apparatus has the following benefits. The apparatus used in the weight loss mode may cause fast and effective lipolysis of the fat cells for fat loss and detoxification, reduction of the appearance of cellulite, increased collagen formation for skin rejuvenation, and increased skin radiance and skin elasticity.

When the apparatus is used in the pain therapy mode, infrared therapy such as FAR-Infrared and LED phototherapy significantly reduces recovery time for wounds, bruises and injuries, increases oxygen and blood flow, improves circulation to the treated area and stimulates antioxidant enzyme release. Phototherapy in this spectrum also promotes muscle relaxation and generally facilitates pain reduction, stimulates fibroblast and collagen production, increases lymphatic drainage, and reduces swelling, inflammation and bruising—bringing relief to athletes, sufferers of arthritis, and those with chronic pain. When the apparatus is used in the visible light mode, the lights are visible and also emit radiation that helps to heal injuries, flatten skin, and smooth wrinkles. The apparatus also enables the user to conveniently select to have the flexible wrap operate in the weight loss mode, the pain therapy mode and the visible light mode.

Although various embodiments of the disclosed apparatus and method for emitting light to a body of a user have been shown and described, modifications may occur to those skilled in the art upon reading the specification. The present application includes such modifications and is limited only by the scope of the claims.

What is claimed is:

1. A light emitting apparatus for use in selected areas of a body of a user, comprising:
a flexible wrap comprising one or more ends configured to detachably connect together;
a plurality of lights arranged on an inner surface of the flexible wrap, the flexible wrap is configured to be wrapped around at least one selected area of the body such that the plurality of lights face the at least one selected area, wherein the plurality of lights are configured to emit a light at a predetermined wavelength to the at least one selected area to perforate one or more fat cells present at the at least one selected area and to drain content of the one or more fat cells when a power is supplied to the plurality of lights; and
a vibration-generating module configured to generate a vibration for stimulating the at least one selected area of the body.

2. The apparatus of claim 1, wherein the plurality of lights comprises a plurality of Light Emitting Diodes (LEDs), wherein each of the plurality of LEDs is configured to emit an infrared radiation at the predetermined wavelength to the at least one selected area of the body for perforating the one or more fat cells at the at least one selected area for draining the content of the one or more fat cells when the power is supplied to the plurality of LEDs.

3. The apparatus of claim 2, wherein the predetermined wavelength is between a range of 630 nm and 635 nm.

4. The apparatus of claim 3 further including a switch configured to enable an activation of the plurality of lights to emit a light at the predetermined wavelength to the at least one selected area of the body.

5. The apparatus of claim 4, wherein the plurality of lights is arranged in an array of lights consisting of 30 columns and 10 rows.

6. The apparatus of claim 5 further comprising a timer circuit configured to enable the plurality of lights to emit the light for one second after every four seconds.

7. The apparatus of claim 6, wherein the vibration-generating module generates the vibration simultaneously with the emission of the light, by the plurality of lights, at the at least one selected area.

8. A light emitting apparatus for use in selected areas of a body of a user, comprising:
a flexible wrap comprising one or more ends configured to detachably connect together;
a plurality of lights arranged on an inner surface of the flexible wrap, the flexible wrap is configured to be wrapped around at least one selected area of the body such that the plurality of lights face the at least one selected area, wherein the plurality of lights are configured to emit a light at a predetermined wavelength at the at least one selected area to perforate one or more fat cells present at the at least one selected area and to drain content of the one or more fat cells when a power is supplied to the plurality of lights, wherein the predetermined wavelength is between a range of 630 nm and 635 nm; and
a vibration-generating module configured to generate a vibration for stimulating the at least one selected area of the body, wherein the vibration-generating module generates the vibration simultaneously with the emission of the light, by the plurality of lights, at the at least one selected area.

9. The light emitting apparatus of claim 8, wherein the plurality of lights comprises a plurality of Light Emitting Diodes (LEDs), wherein each of the plurality of LEDs is configured to emit an infrared radiation at the predetermined wavelength to the at least one selected area of the body for perforating the one or more fat cells at the at least one selected area for draining the content of the one or more fat cells when the power is supplied to the plurality of LEDs.

10. The light emitting apparatus of claim 9 further including a switch configured to enable an activation of the plurality of lights to emit a light at the predetermined wavelength to the at least one selected area of the body.

11. The light emitting apparatus of claim 10, wherein the plurality of lights is arranged in an array of lights consisting of 30 columns and 10 rows.

12. The light emitting apparatus of claim 11 further comprising a timer circuit configured to enable the plurality of lights to emit the light for one second after every four seconds.

13. A method comprising:
wrapping a flexible wrap comprising a plurality of lights around at least one selected area of a body of a user such that the plurality of lights face the at least one selected area; and
supplying power to the flexible wrap to cause each of the plurality lights to emit a light at a predetermined wavelength to the at least one selected area to perforate one or more fat cells at the at least one selected area to drain a content of the one or more fat cells, wherein the first predetermined wavelength is generally between 630 nm and 635 nm;
generating a vibration for stimulation of the at least one selected area of the body.

14. The method of claim 13, wherein the plurality of lights comprises a plurality of Light Emitting Diodes (LEDs), wherein supplying power to the flexible wrap enables the plurality of LEDs to emit infrared radiation at the predetermined wavelength to the at least one selected area of the body to perforate the one or more fat cells to drain the content of the one or more fat cells when the power is supplied to the plurality of LEDs.

15. The method of claim 14, wherein the plurality of LEDs emits infrared light for one second every four seconds.

16. The method of claim 15 further comprising selecting a switch present on the flexible wrap for activating the plurality of lights to emit the light at the predetermined wavelength to the at least one selected area.

17. The method of claim 16 further comprising setting a timer for setting a duration for at least one of emitting the plurality of lights and generating the vibration for stimulating the at least one selected area of the body.

18. The method of claim 17 further comprising generating vibration simultaneously with the emission of the light at the at least one selected area.
